(19) United States
(12) Patent Application Publication
(10) Pub. No.: US 2005/0239757 A1
(43) Pub. Date: Oct. 27, 2005

(54) Hormone treatment of macular degeneration

(76) Inventor: Russell R. Roby, Austin, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/110,199

(22) Filed: Apr. 20, 2005

Related U.S. Application Data
(60) Provisional application No. 60/563,956, filed on Apr. 21, 2004.

(51) Int. Cl. A61K 31/57; A61K 31/56

(52) U.S. Cl. 514/170; 514/177

(57) ABSTRACT

Compositions and/or methods are provided for treating macular degeneration and/or a degenerative ocular condition comprising administering to a human an effective amount of a hormone dilution.

### BACKGROUND

Macular degeneration is a disease that affects the macula, the central portion of the retina, the light-sensitive tissue at the back of the eye. The retina instantly converts light, or an image, into electrical impulses and then sends these impulses to the brain. When macular degeneration develops, a person loses the sharp, central vision needed to see straight ahead and to engage in such activities as reading, sewing and driving. This condition may advance slowly or rapidly and may affect vision in one or both eyes. Age-related macular degeneration is the leading cause of vision loss in Americans sixty years of age and older.

Macular degeneration may occur in two forms, wet and dry. The wet form occurs when abnormal blood vessels behind the retina start to grow under the macula. These new blood vessels tend to be very fragile and often leak blood and fluid. The blood and fluid raise the macula from its normal place at the back of the eye, rapidly damaging the retina. Wet macular degeneration is considered to be advanced and is more severe than the dry form. Dry macular degeneration occurs when the light-sensitive cells in the macula slowly break down, gradually blurring central vision in the affected eye. Over time, as less of the macula functions, central vision in the affected eye may be lost gradually. The dry form of macular degeneration is much more common, occurring in up to 85% of people with intermediate and advanced macular degeneration.

Macular degeneration most often has its onset in middle age, the risk increasing with age. Other risk factors include smoking, obesity, race (increased incidence in Caucasians), positive family history of the disease and female gender.

Macular degeneration may be detected during a comprehensive eye exam that includes a visual acuity test, a dilated eye exam and tonometry. In some cases a fluorescein angiogram may be necessary to diagnose wet macular degeneration.

Once dry macular degeneration reaches the advanced stage, vision loss may not be preventable. Intermediate macular degeneration has been treated with specific high-dose formulations of antioxidants, for example vitamins E and C and zinc.

### SUMMARY

In accordance with teachings of the present disclosure, a method and composition for treating macular degeneration including administering to a human an effective amount of a hormone dilution is provided.

In one embodiment of the disclosure a method of treating macular degeneration with a hormone dilution is provided the method including administering a dilution of progesterone, the dilution configured to be administered sublingually and administering additional dilutions of progesterone as often as necessary to stimulate an effective response.

In another embodiment of the disclosure a composition for the treatment of macular degeneration is provided, the composition including dilute progesterone in a concentration ranging from 0.5 μg/ml to 5 mg/ml.

In various embodiments of the disclosure a composition for the treatment of macular degeneration may be administered as a tablet. In other embodiments of the disclosure the composition may be administered as drops or intradermally by injections or other means. In some embodiments of the disclosure the composition for the treatment of macular degeneration may be administered sublingually.

### DETAILED DESCRIPTION

The present disclosure relates to methods and compositions for treating hormone allergies and their related symptoms and disorders. It also includes methods for diagnosing hormone allergies.

In one embodiment, the disclosure includes dilute hormones for the treatment of symptoms and disorders related to hormone allergy. Normally, the dilute hormone used for treatment is the hormone to which the patient is allergic. Thus, for macular degeneration, the hormone may be, for example, progesterone and/or estrogen. General references to “progesterone” and “estrogen” herein are intended to include any analogs or receptor agonists that are functional in the methods and compositions of the present disclosure. Estrogens may include, without limitation, ethinyl estradiol, β-estradiol and/or all related steroidal compounds. Progesterones may include, without limitation, progestin, allylestrenol, desogestrel, norethindrone and/or norgestrel.

The amount of hormone administered may be the minimal amount needed to alleviate the relevant symptoms. Thus, the appropriate amount may be determined simply by administering to the patient increasing amounts of hormone until alleviation of the symptoms is achieved. While it is possible to administer to the patient an amount of hormone greater than the minimal amount able to achieve alleviation of symptoms, in a specific embodiment, only the minimal amount is administered. Additionally, the minimal amount of hormone able to alleviate symptoms may change during the course of treatment. Such change in minimal dose may also be determined by administering to the patient increasing amounts of hormone until alleviation is achieved. Alternatively, a small dose of hormone may be chosen for administration to most patients. Such dose may be previously determined to be effective in a certain percentage of patients.

Compositions of the present disclosure may be administered with or in a pharmaceutically-acceptable additive. Additives may be selected from the group consisting of carriers, excipients, and diluents. Suitable carriers include buffers such as phosphoric acid, citric acid and other organic acids; antioxidants such as ascorbic acid; low-molecular weight polypeptides; proteins such as serum albumin, gelatin and immunoglobulin; hydrophilic polymers such as polyvinylpyrrolidone; amino acids such as glycine, glutamine, arginine or lysine; monosaccharides such as mannose or dextrin, disaccharides, other carbohydrates; chelating factors such as EDTA; metal ions such as zinc, cobalt or copper; sugar alcohols such as mannitol or sorbitol; salt-forming counter ions such as sodium; and/or non-ionic surfactants such as Tween, Pluronic or polyethylene glycol (PEG). Excipients and diluents may be selected from the group consisting of magnesium stearate, calcium carbonate, starch-gelatin paste, talc, aluminum salt, phenoxyethyl ethanol, water, physiological salt solution, lactose, dextrose, sucrose, sorbitol, mannitol, calcium silicate, cellulose, methyl cellulose, amorphous cellulose, polyvinylpyrolidone, metylhydroxy bezoate, propylhydroxybezoate, and a mineral oil. Other optional components, e.g., stabilizers, buffers, preservatives, flavorings, excipients and the like, may be added.

The hormone may be formulated in any physiologically acceptable carrier. In a specific embodiment, the carrier may be a liquid carrier including an alcohol and oil, or including a saline solution. The volume of carrier may vary, but it may be selected so as to allow delivery of the desired amount of hormone in a small volume, such as one milliliter or less, specifically one hundred microliters. The carrier and volume may be selected based on a variety of factors, including the mode of delivery, the form or concentration in which the hormone is supplied before formulation, and the ability to administer a precise amount of hormone. Although the initial hormone may be supplied in any form, in certain embodiments it may be obtained as an injectable, solubilized hormone that is then further diluted in the carrier.

The hormone may be administered through any effective mode including, without limitation, sublingual administration and intradermal injection. Other possible routes include ocular administration, oral administration, paranteral administration, intradermal injection, subcutaneous injection, intrathyroid injection, and intravenous injection, intranasal, transdermal, transconjunctival, or aerosol mist through any orifice or through the skin. The disclosure additionally contemplates progesterone and/or estrogen delivery using a suitable gene therapy vector.

Compositions of the disclosure may have a form selected from the group consisting of ingestible tablet, buccal tablet, troches, capsule, elixir, suspension, syrup, wafer, pill, granule, powder, cachet, emulsion, liquid, aerosol, soft or hard gelatin capsule, sterilized liquid for injection, sterilized powder and the like.

Many disorders have been associated with changes in the menstrual cycle. Without being limited to any particular mechanism of action, the data disclosed herein may suggest the possibility of hormone allergy and delayed-type hypersensitivity reactions. Accordingly, in some embodiments of the disclosure, hormones may bind to blood proteins such as albumin, globulins, or other proteins, which, after presentation by antigen-presenting cells (APC) to T-helper cells and stimulating Type 2 helper cell response, may result in IgE synthesis and allergic disease. These antibodies reacting with the hormone may induce immune reactions.

In other embodiments of the disclosure, after hormones bind to blood proteins, different lymphocytes may react to this complex and induce lymphocyte proliferation and cytokine production, resulting in Type IV allergic reaction or delayed typed hypersensitivity.

Thus, a number of disorders may be ameliorated, treated, or prevented by determining the presence of hormone allergy and, if present, administering a desensitizing dose of the hormone to the subject. The present disclosure relates to ameliorating, treating, and/or preventing macular degeneration and/or any degenerative ocular condition, disorder, or disease (collectively “condition”) caused at least in part by a sensitivity or allergic reaction to a hormone.

According to some embodiments, a method and composition for treatment of macular degeneration using dilute hormone dilutions is provided. Observations that lead to and are a part of the present disclosure, may suggest the possibility of an allergic reaction to the steroid hormone progesterone as a possible cause of macular degeneration and other disorders.

One aspect of the present disclosure includes a previously unrecognized treatment for macular degeneration that involves desensitizing a body's response to its own innate hormones. The treatment may be applied to any mammal including humans. In one embodiment, the mammal is a female with a clinical history of macular degeneration.

The therapeutic effect of therapy, e.g., amelioration, treatment, and/or prevention of macular degeneration may be assessed by any means including, without limitation, vision tests and eye examination. Vision tests may include, without limitation, visual acuity tests (e.g., eye charts, Snellen charts, and/or Amsler grids), wherein any reduction in the rate of loss of visual acuity may indicate a therapeutic effect. The rate of loss may be an empirically determined rate of loss or an expected rate of loss due to the presence of one or more risk factors. Eye examination may include, without limitation, an angiography to test for the presence and/or integrity of blood vessels in the retina and/or an evaluation of the presence and number of drusen—tiny yellow deposits in the retina.

While hormones may fluctuate throughout the menstrual cycle, treatment is not limited to any specific point in the menstrual cycle. In one embodiment, however, dilute solutions of progesterone are administered sublingually, every day or every other day, as needed, until there is an alleviation of a patient's clinical symptoms. In some embodiments of the disclosure, macular degeneration may be ameliorated, treated, or prevented by administering low doses of progesterone and/or estrogen sufficient to attenuate a progesterone and/or estrogen allergy. These dilute formulations may be very similar to the type of dilutions that an allergist typically uses when treating allergic symptoms from external substances, or allergens, which are foreign to the body. However, in treating a patient with hormone allergy, instead of desensitizing the patient to a foreign substance, the patient is desensitized to his or her own innate hormone(s).

In accordance with another aspect of the present disclosure, dilutions of a hormone solution, such as progesterone, are used to treat macular degeneration. A hormone dilution ranging in concentration from 5 mg/ml to 0.5 μg/ml is administered sublingually. Although a 10% solution is preferable for some patients, the strength of the dilution selected for treatment may be based on the severity of the patient's symptoms and prior treatment history. The amount, frequency and strength of the hormone dilution may be varied depending on severity of symptoms and on response achieved. The dilution may be in the form of a liquid solution that may be a suspension or drops or the dilution may be in the form of a sublingual tablet or any other oral formulation, liquid or solid, suitable for administration of hormone dilutions.

In an alternative embodiment of the disclosure, the route of administration may be intradermal. In accordance with a further aspect of this disclosure a dilute progesterone solution (concentration 5 mg/ml to 0.5 μg/ml) or a dilute estrogen solution (concentration 5 mg/ml to 0.5 μg/ml) may be administered to treat hormone allergy symptoms in females. A solution ranging from approximately a 1% dilution to a 20% dilution may be used or any other dilution suitable for achieving the desired clinical effect.

A composition of the present disclosure may include a standard solution of aqueous progesterone, or any other indicated steroid hormone, diluted with normal saline to achieve concentrations of a desirable concentration. The strength of a dilution selected for treatment may be based on severity of the patient's symptoms and prior treatment history. This selection methodology may be similar to that used in treatments with foreign allergens and appropriate selections for an individual patient will be apparent to one skilled in the art.

In one embodiment of the disclosure, 0.1 cc or a comparable sublingual tablet formed of a 10% dilution of progesterone is administered sublingually every day for sixty days. The frequency of administration may be increased or decreased as required, to achieve a desired treatment response. The strength of the hormone dilution selected for treatment may also be varied depending on severity of symptoms and on response achieved.

Before dilute hormone therapy is administered, baseline levels of serum progesterone antibodies may be measured. Response to therapy is measured by serum progesterone antibodies that may be assayed at any point during or after therapy. Response to therapy is also measured by improved vision or stabilization of macular degeneration on direct examination of the retina. Intracellular cytokine assays may also be performed pre- and post-therapy to measure response rates to therapy.

In an alternative embodiment of this disclosure, the dilution may be administered intradermally for instance, in patients who may have no response to sublingual drops or patients who are unable to use the sublingual delivery method.

Certain embodiments of the present disclosure are additionally related to methods of diagnosing hormone allergy in patients. Because the presence of immunoglobulin E (IgE) is required for a Type 1 allergic reaction, detection of elevated anti-hormone IgE may be indicative of a Type 1 hormone allergy. Presence of immunoglobulin G (IgG), immunoglobulin M (IgM) or immunoglobulin A (IgA) may be indicative of a Type 2 or 3 hormone allergy. An assay for an immunoglobulin (Ig) may be particularly useful in patients exhibiting the symptoms or disorders described herein. Detection of elevated anti-hormone immunoglobin (Ig) provides a clue as to which hormone may be responsible for the symptoms or disorder, thus guiding treatment. Failure to detect elevated levels of anti-hormone Ig may indicate that the symptoms or disorder are caused by something other than hormone allergy, such as a different autoimmune disorder.

In some embodiments diagnosis may focus on detection of anti-hormone IgE because of its role in rapid allergic responses.

In patients that exhibit elevated anti-hormone antibodies, e.g., IgE, as well as patients that have inconclusive results or do not exhibit elevated antibody levels, a decrease in anti-hormone antibodies, particularly IgE, after treatment may still be indicative of a hormone allergy. This is particularly true if the patient additionally exhibits improvement in a hormone allergy-related symptom or disease after treatment. Thus, although many patients with hormone allergy may be identified by high levels of anti-hormone antibodies, this method may not be suitable for all patients. For example, patients who produce low amounts of antibodies overall as compared to normal patients may require diagnosis by this second method.

### EXAMPLES

### Example 1

### Dilution Protocol

Progesterone USP 50 mg/ml (Schein Laboratories, Florham, N.J.) is diluted with physiologically-compatible (normal) saline to produce the progesterone dilutions used in treatments. The initial progesterone is suspended in sesame oil. Therefore, to achieve an even suspension, the vial must be vigorously shaken at each stage of the initial preparation and before use of each vial. The first dilution is made by adding 0.5 ml of progesterone to 4.5 ml normal saline. This results in a 1:10 dilution of progesterone (progesterone 5 mg/ml) which is labeled “PROG 1.” After vigorously shaking the PROG 1 vial, 0.5 ml is withdrawn and injected into the next vial of 4.5 ml of normal saline. This results in a 1:100 dilution of Progesterone (0.5 mg/ml, “PROG 2”). To produce the next dilution, a vial of PROG 2 is immediately withdraw 0.5 ml and injected into the next vial of 4.5 ml of normal saline. This results in a 1:1000 dilution of Progesterone (50 μg/ml “PROG 3”). These steps are repeated until there are five serial dilutions labeled “PROG 1” through “PROG 5.” (See Table 1). A milligram (mg) is defined as 1/1000 or 10⁻³ of a gram. A microgram (μg) is defined as 1/1,000,000 or 10⁻⁶ of a gram.

TABLE 1
Progesterone Dilutions

| | Progesterone | | Dosage Used |
|---|---|---|---|
| Label | Concentration | Dilution | (0.1 mL) |
| PROG 1 | 5 mg/ml | 10⁻¹ | 0.5 mg |
| PROG 2 | 0.5 mg/ml | 10⁻² | 0.05 mg |
| PROG 3 | 50 μg/ml | 10⁻³ | 5 μg |
| PROG 4 | 5 μg/ml | 10⁻⁴ | 0.5 μg |
| PROG 5 | 0.5 μg/ml | 10⁻⁵ | 0.05 μg |

### Example 2

### Blood

Hormone levels were examined as part of routine work-ups of adult allergy patients. Tests for hormone antibodies were initiated when prick and sublingual tests with hormones resulted in changes in symptoms. Over a three-year period, 368 female patients were tested for hormone antibodies.

Since progesterone was the hormone most commonly associated with symptom changes when used as a test antigen, tests conducted over the first two years were only directed to IgM and IgG antibodies to progesterone. Blood samples were taken from 270 female patients who experienced a change in symptoms associated with their menstrual cycle. The women were 24-47 years of age. Blood samples were obtained from 500 healthy control subjects by a commercial lab (Immunosciences Lab., Inc., Beverly Hills, Ca.). During the last year, tests were performed for IgE against estrogen and progesterone using 32 healthy patients as controls and 98 patients who noted perimenstrual symptom changes.

### Example 3

### Hormones, Antibodies and Reagents

Human serum albumin (HSA), bovine serum albumin (BSA), estradiol-BSA and progesterone-BSA, phosphate buffered saline (PBS) and substrate (BNPP) were purchased from Sigma chemicals (St. Louis, Mo., USA).

Alkaline phosphatase-labeled goat anti-human IgG, IgM and IgE were purchased from KPL (Gaithersburg, Md., USA).

### Example 4

### ELISA for Estrogen and Progesterone Antibody

Enzyme-linked immunosorbent assay (ELISA) was used for testing antibodies against estrogen and progesterone in the sera of patients with premenstrual asthma and with control subjects. Different rows of microtiter plates (Costar) were coated either with 100 μl of BSA concentration of 10 μg/mL or 100 μl of estrogen-BSA or progesterone-BSA optimal concentration of 10 μg/mL in 0.1 m carbonate-bicarbonate buffer (pH 9.5). Plates were incubated overnight at 4° C. and then washed three times with 200 ml of Tris-buffered saline (TBS) containing 0.05% Tween 20, pH 7.4. The non-specific binding of immunoglobulins (Igs) was prevented by adding a mixture of 1.5% bovine serum albumin (BSA) and 1.5% gelatin in TBS and then incubating this mixture for 2 h at room temperature and then overnight at 4° C. Plates were washed with PBS-Tween 20 and then 100 μl of control or patient's serum was added to duplicate wells coated either with BSA alone or with estrogen or progesterone bound to BSA. The optimal dilution of serum was determined by checkerboard dilution and found to be 1:100 for IgG and IgM and 1:2 for IgE. Plates were incubated for 2 h (for IgG and IgM) and overnight (for IgE), and then washed four times with PBS-Tween 20. Alkaline-phosphatase-conjugated goat anti-human IgG, IgM or IgE F(ab′)₂ fragment at optimal dilution of 1:700 (IgG); 1:500 (IgM) and 1:250 (IgE) was added to corresponding wells. The plates were then incubated for an additional 2 h at room temperature. After washing five times with TBS-Tween buffer, the enzyme reaction was started by adding 100 μl of para-nitrophenylphosphate in 0.1 mL of diethanolamine buffer (1 mg/ml) containing 1 mM MgCl₂ and sodium azide, pH 9.8. The reaction was stopped 45 minutes later with 50 μl of 1 N NaOH. The optical density was read at 405 nm (OD₄₀₅) with a microtiter reader. Optical densities coated with BSA alone were not more than 0.2. However, this non-specific O.D. was subtracted from wells coated with estrogen or progesterone bound to BSA.

In the next step, for construction of standard curve and conversion of optical densities to ELISA values, the following three calibrators were used:
Calibrator I—Serum from patient with no known allergy giving optical density of 0.2-0.4 at 405 nm when serum was diluted at 1:100. This control was assigned an ELISA value of 10.
Calibrator II—Serum from patient with hormone allergy giving optical density of 0.41-1.0 when diluted at 1:100. This control was assigned an ELISA value of 20.
Calibrator III—Serum from patient with hormone allergy giving optical density greater than 1.0 when diluted at 1:100. This control was assigned an ELISA value of 80.
   The following controls were used for these calibrations:
Negative control serum—Serum from healthy individual, which, at dilution of 1:100 will not give an O.D. greater than 0.3 when measured at 405 nm.
Positive control serum—Serum from patient with hormone allergy, which, at dilution of 1:100 will not give an O.D. greater than 0.7 when measured at 405 nm.
   These calibrations were used to:
Construct a curve by plotting the mean absorbance obtained for each calibrator against its concentration on a linear graph paper, with absorbance on the vertical (y) axis and concentration on the horizontal (x) axis; and
Determine the corresponding concentration of gluten antibody from the standard curve using the mean absorbance value for each control and unknown samples.

The ELISA value of the test specimens was calculated using Equation (1):

$${EV}_{TS} = \frac{CV \times {A}_{TS}}{{A}_{C}} \qquad ( 1 )$$

wherein EV_{TS} is the ELISA value of the test specimen, CV is the calibrator value, A_{TS} is the absorbance of the test specimen, and A_{C} is the absorbance of the calibrator. This calculation was performed automatically by the ELISA reader.

### Example 5

### Inter- and Intra-Assay Precision

The inter-assay reproducibility was determined by assaying eight different samples in duplicate using the hormone antibody ELISA assay on each 5 consecutive days. Each assay was performed using freshly prepared reagents. The % C.V. for samples with high O.D. (2.0 or greater) was between 5-8%, and for the samples with optical densities of 1.0 or less, between 10-20%.

The intra-assay reproducibility was determined by assaying eight different samples, eight different times simultaneously. Each assay was performed using freshly prepared reagents. The % C.V. for samples with O.D. between 1.0-2.5 was less than 10%, and for the samples with optical densities of 0.1-0.5, less than 20%.

### Example 6

### Specificity of Hormone Antibodies

Absorption of sera with specific and non-specific antigens was used to demonstrate that these anti-hormone antibodies are specific. For this, microtiter plates were coated with hormones and blocked by the addition of 2% BSA in PBST. 100 μl of serum diluent buffer was added to all wells. Then estrogen-BSA, progesterone-BSA, BSA, myelin basic protein (MBP), and human serum albumin (HSA) starting at concentration of 1 mg/mL was added to the second rows of 1-8 strips and titered down the column in ½ log dilution. After a 60-minute incubation, 100 μl serum anti-estrogen or anti-progesterone was added to all wells. Addition of enzyme-labeled second antibody after incubation and washing resulted in color development, which was measured at 405 nm. Results were calculated as a percentage of inhibition in antigen-antibody reaction.

To examine whether antibodies to estrogen or progesterone are specific or cross-reactive, competition ELISA was performed by adding specific and non-specific antigens in liquid phase and examined prevention of serum antibody binding to the antigen in solid phase. Results summarized in Tables 2-3 showed that BSA, HSA and MBP did not absorb the serum IgG and IgE antibodies when they were added to the liquid phase. But addition of estrogen-BSA (En) or progesterone-BSA (Pn) significantly absorbed the IgG and IgE antibodies. This inhibition of anti-estrogen binding to estrogen by estrogen-BSA in liquid phase was between 52-67% and by progesterone-BSA was between 41-52%. For IgE anti-estrogen this inhibition by estrogen-BSA was between 54-62%, and with progesterone-BSA from 37-43%. These results indicate that while antibodies against hormones are specific, they may be cross-reacting between estrogen and progesterone. This cross-reaction between estrogen and progesterone antibodies may be due to structural similarities between these two hormones. Similar results were obtained when progesterone antibodies were absorbed with estrogen or progesterone bound to BSA.

TABLE 2
Serum Anti-Estrogen Level and Inhibition with Specific and
Non-Specific Antigens

| | IgG level | | | | | |
|---|---|---|---|---|---|---|
| | (Percent Inhibition after Absorption | | | | | |
| | with 250 μg/mL) | | | | | |
| | Before | | | | | |
| Sample | Abs'n | BSA | HSA | MBP | En | Pn |
| 1 | 1.83 | 1.79 | 1.81 | 1.67 | 0.61 | 0.87 |
| Percent | — | (NS) | (NS) | (NS) | (67%) | (52%) |
| Inhibition | | | | | | |
| 2 | 1.25 | 1.13 | 1.19 | 1.13 | 0.52 | 0.69 |
| Percent | — | (NS) | (NS) | (NS) | (59%) | (45%) |
| Inhibition | | | | | | |
| 3 | 0.76 | 0.81 | 0.78 | 0.68 | 0.37 | 0.45 |
| Percent | — | (NS) | (NS) | (NS) | (52%) | (41%) |
| Inhibition | | | | | | |

NS = non-significant;
Abs'n = absorption;
BSA = bovine serum albumin;
HSA = human serum albumin;
MBP = myelin basic protein;
En = Estrogen-BSA;
Pn = Progesterone-BSA

TABLE 3
Serum Anti-Estrogen Level and Inhibition with Specific and
Non-Specific Antigens

| | IgE level | | | | | |
|---|---|---|---|---|---|---|
| | (Percent Inhibition after Absorption | | | | | |
| | with 250 μg/mL) | | | | | |
| | Before | | | | | |
| Sample | Abs'n | BSA | HSA | MBP | En | Pn |
| 1 | 2.15 | 2.24 | 2.11 | 2.05 | 0.96 | 1.35 |
| Percent | — | (NS) | (NS) | (NS) | (55%) | (37%) |
| Inhibition | | | | | | |
| 2 | 1.66 | 1.53 | 1.59 | 1.73 | 0.77 | 0.98 |
| Percent | — | (NS) | (NS) | (NS) | (54%) | (41&) |
| Inhibition | | | | | | |
| 3 | 1.34 | 1.26 | 1.21 | 1.11 | 0.51 | 0.76 |
| Percent | — | (NS) | (NS) | (NS) | (62%) | (43%) |
| Inhibition | | | | | | |

NS = non-significant;
Abs'n = absorption;
BSA = bovine serum albumin;
HSA = human serum albumin;
MBP = myelin basic protein;
En = Estrogen-BSA;
Pn = Progesterone-BSA

### Example 7

### Results

IgG and IgM against Progesterone. Of 270 patients tested, 142 had high levels of IgG, IgM, or both when compared to the 500 controls set up by Immunoscience Labs.

IgE against Estrogen. Sera from 19 healthy subjects were analyzed using ELISA assays for IgE against estrogen. The mean±SD was 13.4±2.3. Sera from 15 patients were analyzed, with a mean assay of 26.8±15.6. Student's-t one-tailed test gave a highly significant difference of patients from control (p≦0.0009).

IgE against Progesterone. Sera from 13 healthy subjects were analyzed using ELISA assays for IgE against progesterone. The mean±SD was 17.31±3.0. Sera from 83 patients were 23.3±7.1. Student's-t one-tailed test gave a highly significant difference of patients from control (p≦0.000003).

In spite of these highly significant differences between healthy and clinical populations of subjects, there are notable opportunities for IgE normal versus clinical symptomatic misclassifications for both hormone antigens. Although all estrogen controls were within normal range, 2 of 15 patients were within normal range. For progesterone, 3 of 13 control individuals were marginally above normal range. Similarly, 44 of 83 patients overlapped normal range but were predominantly greater than one standard deviation above the mean. While the difference between control and patients was striking, there was no unequivocal boundary between “normal” vs. “abnormal” levels of IgE for either estrogen or progesterone.

### Example 8

### Clinical Results

An adult human female presented with macular degeneration. She had visual acuity of 20/200 in each eye. After the use of sublingual progesterone, one application, her vision improved to 20/100 and 20/40. On her return visit two weeks later she reported that her ophthalmologist had told her she was “remarkably improved”. She was evaluated for hormone allergy therapy by assessing her anti-hormone antibodies and found to have elevated levels of IgE, IgG, and IgM antibodies to progesterone.

The subject then received progesterone 1:10 sublingual drops which she continues to use. There has been continued improvement in her vision and her macular degeneration is in good control.

Although embodiments of the present invention have been described in detail, it should be understood that various changes, substitutions and alternations can be made herein without departing from the spirit and scope of the invention as illustrated by the following claims.

1. A method for treating macular degeneration comprising administering to a human an effective amount of a hormone dilution.

2. The method of claim 1 wherein the hormone dilution comprises a steroid hormone solution with a concentration from about 5 mg/ml to about 0.5 μg/ml.

3. The method of claim 1, wherein the hormone dilution comprises a progesterone, an estrogen, or a progesterone and an estrogen.

4. The method of claim 1 further comprising the concentration of the hormone solution selected from the group consisting of 5 mg/ml, 0.5 mg/ml, 50 μg/ml, 5 μg/ml, and 0.5 μg/ml.

5. The method of claim 1 wherein the hormone dilution comprises a progesterone dilution of 10%.

6. The method of claim 6 further comprising the amount of progesterone administered ranges from between approximately 0.5 mg to approximately 0.05 μg per dose.

7. The method of claim 1 wherein the hormone dilution is administered sublingually.

8. The method of claim 1 wherein the hormone dilution is administered in a sublingual tablet.

9. The method of claim 1 wherein the hormone dilution is administered in sublingual drops.

10. The method of claim 1 wherein the hormone dilution is administered intradermally.

11. A method of treating macular degeneration with a hormone dilution comprising:
administering a dilution of progesterone, the dilution configured to be administered sublingually; and
administering additional dilutions of progesterone as often as necessary to stimulate an effective response.

12. The method of claim 11 wherein the hormone dilution comprises drops.

13. The method of claim 11 wherein the hormone dilution comprises a sublingual tablet.

14. A method of treating a degenerative disease of the eye with a hormone dilution comprising:
administering a dilution of progesterone, the dilution configured to be administered sublingually; and
administering additional dilutions of progesterone as often as necessary to stimulate an effective response.

15. A method of treating macular degeneration using sublingual progesterone dilutions comprising administering the progesterone dilution once a day, the progesterone dilution comprising a sublingual tablet or solution formed of 10% progesterone.

16. The method of claim 15, wherein the daily progesterone dilution administration continues for approximately sixty days.

17. The method of claim 15, wherein serum anti-progesterone antibodies are measured.

18. The method of claim 15 wherein intracellular cytokines are measured.

19. A composition for treating macular degeneration comprising dilute progesterone in a concentration ranging from approximately 0.05 μg/ml to approximately 5 mg/ml.

20. The composition of claim 19 wherein the progesterone dilution comprises a sublingual tablet.

21. The composition of claim 19 wherein the hormone dilution comprises drops.

22. A composition for treating macular degeneration comprising a concentration of progesterone selected from the group consisting of 5 mg/ml, 0.5 mg/ml, 50 μg/ml, 5 μg/ml, and 0.5 μg/ml.

23. The composition of claim 22 wherein the progesterone dilution comprises a sublingual tablet.

24. The composition of claim 22 wherein the hormone dilution comprises drops.

25. The composition of claim 22 wherein the progesterone dilution is administered intradermally.
